(19) United States
(12) Patent Application Publication
(10) Pub. No.: US 2025/0125058 A1
(43) Pub. Date: Apr. 17, 2025

(54) SAFETY EVENT SURVEILLANCE AND ANALYSIS SYSTEM FOR HEALTHCARE ORGANIZATION

(71) Applicant: HILL-ROM SERVICES, INC., Batesville, IN (US)

(72) Inventors: Angela WILLIAMS, Batesville, IN (US); Autumn THOMPSON, Batesville, IN (US); Daniel C. SHIRLEY, Batesville, IN (US)

(21) Appl. No.: 18/910,898

(22) Filed: Oct. 9, 2024

Related U.S. Application Data
(60) Provisional application No. 63/589,419, filed on Oct. 11, 2023.

(51) Int. Cl. G16H 50/30; G16H 15/00; G16H 40/67

(52) U.S. Cl. CPC G16H 50/30; G16H 15/00; G16H 40/67

(57) ABSTRACT

A method for evaluating risk associated with a patient comprises the steps of using a sensor or an assessing device for detecting a safety event involving the patient, using the sensor or the assessing device for acquiring data for identifying and investigating the safety event, operating a risk evaluator to determine an output of the risk associated with the patient, and transmitting the output to a user device.

This application claims priority to U.S. Provisional Patent Application No. 63/589,419, filed Oct. 11, 2023, which is herein incorporated by reference in its entirety.

### BACKGROUND

The present disclosure relates to the use of systems and methods to evaluate risk and improve patient care. More specifically, the present disclosure is directed to a system or method for identifying, investigating, tracking, and predicting safety events in a healthcare system.

Healthcare systems throughout continue to struggle with identifying and responding to patient safety events in a timely and effective manner. With a growing number of healthcare systems committing to optimizing patient safety, there is a clear need for the healthcare systems to incorporate bedside technology within a patient safety surveillance and management framework in order to streamline the workload. Such incorporation can result in the assimilation of accurate information essential to patient safety. While there continues to be development in the field of medical devices with connected capabilities, there persists a deficiency in both the analytics and insights that are capable of informing patient safety. The amount of time required to manually harvest data, coordinate communications, conduct investigations, and develop mitigating action plans places a huge burden on staff in an already taxed healthcare workforce.

Investigating reports of safety events and conducting investigations for root-cause analysis requires an enormous amount of manual work and time. Coordinating investigations and developing a consensus on the root cause of an occurrence often takes weeks to months instead of days. However, a shorter timeframe is ideal for protecting the patient population from future similar events. Therefore, expeditiously conducting these investigations as efficiently and accurately as possible is vital to ensuring safety and reducing pressure on healthcare staff.

Leveraging smart care technology in combination with reporting software to streamline the investigation and reporting of safety events is useful to improve the healthcare system. Such a streamlined process can result in a reduction of serious safety events and sentinel events, reduce time to safety event classification, maximize efficiency and effectiveness of quality and safety human capital, eliminate déjà vu events, capitalize on near misses and optimize the return of investment on smart care technologies, increase patient satisfaction, and increase caregiver satisfaction, staff recruitment and retention.

### SUMMARY

The present disclosure includes one or more of the features recited in the appended claims and/or the following features, which alone or in any combination, may comprise patentable subject matter.

In a first aspect, the present disclosure is directed to a method for evaluating risk associated with a patient comprising the steps of detecting, using a sensor or an assessing device, a safety event involving the patient, acquiring, using the sensor or the assessing device, data for identifying and investigating the safety event, receiving and processing the data to evaluate the risk associated with patient care, operating a module of a risk evaluator to use the data acquired to determine an output of the risk associated with the patient, and transmitting the output to a user device.

In some embodiments, operating the module of the risk evaluator comprises operating a data aggregator module. Operating the operating a data aggregator module comprises the steps of determining if the safety event caused harm to the patient, prompting a user to log a time of occurrence of the safety event, identifying event-related data acquired from the sensor or the assessing device about the patient during a given time period based on the time of occurrence of the safety event, and prompting the user to complete an investigation into the safety event.

In some embodiments, operating the module of the risk evaluator comprises operating a partial data flow. Operating the partial data flow comprises the steps of determining if the safety event caused harm to the patient, prompting a user to log a time of occurrence of the safety event, partially auto-filling an incident investigation report based on the data received from the sensor and the assessing device, identifying event- related data about the patient during a given time period based on the time of occurrence of the safety event, and prompting the user to complete an investigation into the safety event by providing the partially auto-filled incident investigation report.

In some embodiments, operating the module of the risk evaluator comprises operating a classifier. Operating the classifier comprises the steps of determining if the safety event caused harm to the patient, prompting a user to log a time of occurrence of the safety event, utilizing an AI-based operation to determine a classification of the safety event, partially auto-filling an incident investigation report with the data received from the sensor and the assessing device, and prompting the user to complete an investigation into the safety event by providing the partially-filled incident investigation report. In some embodiments, the method further comprises utilizing the AI-based operation to determine a level of patient harm. In some embodiments, the AI-based operation is configured to access data from a patient-controlled analgesia (PCA) pump, a respiratory equipment, a surveillance device, a fluid management device, a bring your own device (BYOD), a patient support apparatus, or a real-time locating system (RTLS). In some embodiments, the AI-based operation is configured to access a root cause analysis (RCA) training set to determine the classification of a safety event.

In some embodiments, operating the module of the risk evaluator comprises operating a root cause determiner. Operating the root cause determiner comprises the steps of determining if the safety event caused harm to the patient, prompting a user to log a time of occurrence of the safety event, utilizing an AI-based operation to determine a root-cause of the safety event, partially auto-filling an incident investigation report based on the data received from the sensor and the assessing device, and prompting the user to complete an investigation into the safety event by providing the partially-autofilled incident investigation report.

In some embodiments, the method further comprises utilizing AI-based operation to determine a level of patient harm. In some embodiments, the method further comprises utilizing AI-based operation to determine a classification of the safety event. In some embodiments, the AI-based operation is configured to access data from a PCA pump, a respiratory equipment, a surveillance device, a fluid management device, a BYOD, a patient support apparatus, or a RTLS. In some embodiments, the AI-based operation is configured to access a RCA training set to determine the root-cause of a safety event.

In some embodiments, operating the module of the risk evaluator comprises operating a recommender module. Operating the recommender module comprises the steps of determining if the safety event caused harm to the patient, prompting a user to log a time of occurrence of the safety event, utilizing an AI-based operation to determine a root-cause of the safety event, utilizing the AI-based operation to write a description about the root-cause of the safety event, partially auto-filling an incident investigation report with the data received from the sensor and the assessing device, and recommending an action plan to the user based on the root-cause determination made by the AI-based operation. In some embodiments, the AI-based operation is configured to access data from a patient-controlled analgesia (PCA) pump, a respiratory equipment, a surveillance device, a fluid management device, a bring your own device (BYOD), a patient support apparatus, or a real-time locating system (RTLS). In some embodiments, the AI-based operation is configured to access a root cause analysis (RCA) training set to classify the safety event. In some embodiments, the AI-based operation is configured to access a RCA training set before recommending the action plan.

In some embodiments, operating the module of the risk evaluator comprises operating an AI predictor module. Operating the AI predictor module comprises the steps of installing and connecting an AI system to the accessing device, training and verifying the AI system's ability to predict a second safety event, utilizing the AI system to predict the second safety event, and transmitting an alert to the user to prevent an occurrence of the second safety event. In some embodiments, the AI system is configured to access data from a patient-controlled analgesia (PCA) pump, a respiratory equipment, a surveillance device, a fluid management device, a bring your own device (BYOD), a patient support apparatus, or a real-time locating system (RTLS). In some embodiments, the AI system utilizes a dataset for training. In some embodiments, the datasets comprise EMR data, root cause analysis (RCA) data, or other data analytics.

In some embodiments, operating the module of the risk evaluator comprises operating an investigation trigger module. Operating the investigation trigger module comprises the steps of training an AI system to learn about data elements that contribute to the safety event, identifying a data abnormality from the data received from the sensor, and assessing device, completing an incident investigation process and generating a safety event report, and transmitting the safety event report to the user device. In some embodiments, the AI system is configured to access data from a patient-controlled analgesia (PCA) pump, a respiratory equipment, a surveillance device, a fluid management device, a bring your own device (BYOD), a patient support apparatus, or a real-time locating system (RTLS). In some embodiments, the AI system is configured to utilize a training data set for identifying the data abnormality.

In a second aspect, the disclosure is directed to a system for evaluating risk associated with a patient. The system comprises an assessing device to capture data for identifying and investigating a safety event, a risk evaluator including a controller and a memory device, the controller operable to receive and process data from the assessing device, the controller further operable to evaluate the risk associated with patient care by operating a module based on the data and a training dataset in the memory device, and a user device including a display, the display illustrating risk associated with patient-care.

In some embodiments, the accessing device comprises a patient-controlled analgesia (PCA) pump, a respiratory equipment, a surveillance device, a fluid management device, a bring your own device (BYOD), a patient support apparatus, or a real-time locating system (RTLS). In some embodiments, the risk evaluator is operable to use data from a patient electronic medical record (EMR), a patient support apparatus, and a RTLS to autofill an incident investigation report. In some embodiments, the risk evaluator is operable to implement a data aggregator module, a partial data flow module, a classifier module, a root-cause determiner module, a recommender module, an AI predictor module, or an investigation trigger module.

In some embodiments, the risk evaluator is operable to implement a data aggregator module configured to access data about the patient from the assessing device during a given time period based on a time of occurrence of the safety event.

In some embodiments, the risk evaluator is operable to implement a partial data flow module configured to access data about the patient from the assessing device during a given time period based on a time of occurrence of the safety event, and partially auto-fill an incident investigation report with the data.

In some embodiments, the risk evaluator further comprises a AI system, and wherein the risk evaluator is operable to implement a classifier module configured to access data about the patient from the assessing device during a given time period based on a time of occurrence of the safety event, utilize an AI-based operation to determine a classification of the safety event, and partially auto-fill an incident investigation report with the data.

In some embodiments, the risk evaluator further comprises a AI system, and wherein the risk evaluator is operable to implement a root-cause determiner module configured to access data about the patient from the assessing device during a given time period based on a time of occurrence of the safety event, utilize an AI-based operation to determine a root-cause of the safety event, and partially auto-fill an incident investigation report with the data.

In some embodiments, the risk evaluator further comprises an AI system, and wherein the risk evaluator is operable to implement a recommender module configured to access data about the patient from the assessing device during a given time period based on a time of occurrence of the safety event, utilize an AI-based operation to determine a root-cause of the safety event, partially auto-fill an incident investigation report with the data, and transmit a recommended action plan to the user device.

In some embodiments, the risk evaluator further comprises an AI system connected to the assessing device, and wherein the risk evaluator is operable to implement an AI predictor module configured to predict a second safety event and transmit the prediction to the user device.

In some embodiments, the risk evaluator further comprises an AI system, and wherein the risk evaluator is operable to implement an investigation trigger module configured to identify a data abnormality from the data received from the assessing device, complete an incident investigation process, generate a safety event report, and transmit a the safety report to the user device.

Additional features, which alone or in combination with any other feature(s), such as those listed above and/or those listed in the claims, can comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is an illustration of a risk evaluation system comprising different subsystems.

FIG. 2 is an illustration of one embodiment of a risk evaluator.

FIG. 3 is a flowchart illustrating a set of operations performed by a data aggregator module comprised in the risk evaluation system.

FIG. 4 is a flowchart illustrating a set of operations performed by a partial data flow module comprised in the risk evaluation system.

FIG. 5 is a flowchart illustrating a set of operations performed by a classifier module comprised in the risk evaluation system.

FIG. 6 is a flowchart illustrating a set of operations performed by a root-cause determiner module comprised in the risk evaluation system.

FIG. 7 is a flowchart illustrating a set of operations performed by a recommender module comprised in the risk evaluation system.

FIG. 8 is a flowchart illustrating a set of operations performed by an AI predictor module comprised in the risk evaluation system.

FIG. 9 is a flowchart illustrating a set of operations performed by an investigation trigger module comprised in the risk evaluation system.

### DETAILED DESCRIPTION

According to the present disclosure, a risk evaluation system 10 can provide real-time insight into the safety status of a healthcare system. The risk evaluation system 10 can be operated or implemented for detecting, reporting, analyzing, and/or tracking patient safety events, outcomes, and/or healthcare system organizational status. The risk evaluation system 10 can utilize the data 12 captured by assessing devices 14 to inform safety event identification, investigation, classification, action plan development, action plan implementation, and loop closure processes. The risk evaluation system 10 can also be used to report any required metrics mandated by local state and national organizations. Machine learning and/or artificial intelligence can be used to predict high-risk conditions for repeat events based on the data 12 captured by the assessing devices 14. The risk evaluation system 10 can enable system-based comprehensive surveillance of safety concerns within the healthcare system. Smart technologies enable continuous monitoring of patient clinical status and satisfaction, therapies, and staff location. This real-time information can be imported into a patient's medical record by the risk evaluation system 10. The risk evaluation system 10 can be utilized for short-term post-event analysis and for long-term post-event analysis.

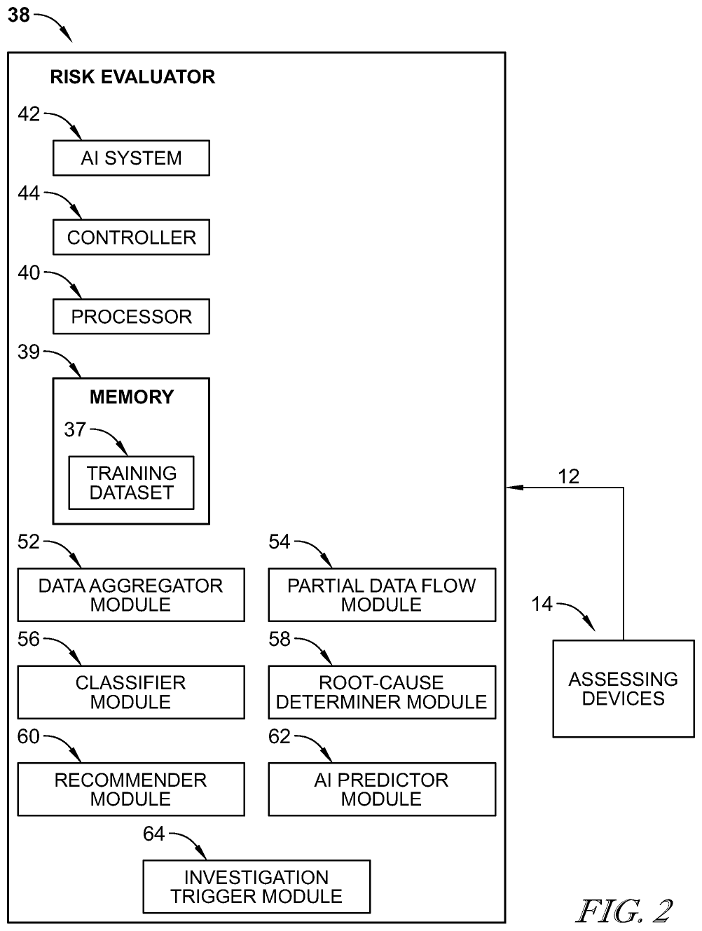

The risk evaluation system 10 comprises a risk evaluator 38 that can be a control system 38, a special purpose device 38, or a controller associated with another component of the healthcare system. The risk evaluator 38 can operate independently or operate in conjunction with a different device in the healthcare system. FIG. 2 illustrates one embodiment of the risk evaluator 38. The risk evaluator 38 can receive information from the assessing devices 14 to evaluate the risk associated with patient care. The risk evaluator 38 comprises one or more training data sets 37 stored in a memory device 39 and executed by one or more processors 40. A controller 44 can execute one or more modules 52, 54, 56, 58, 60, 62, 64, which are subsystems of the risk evaluator 38.

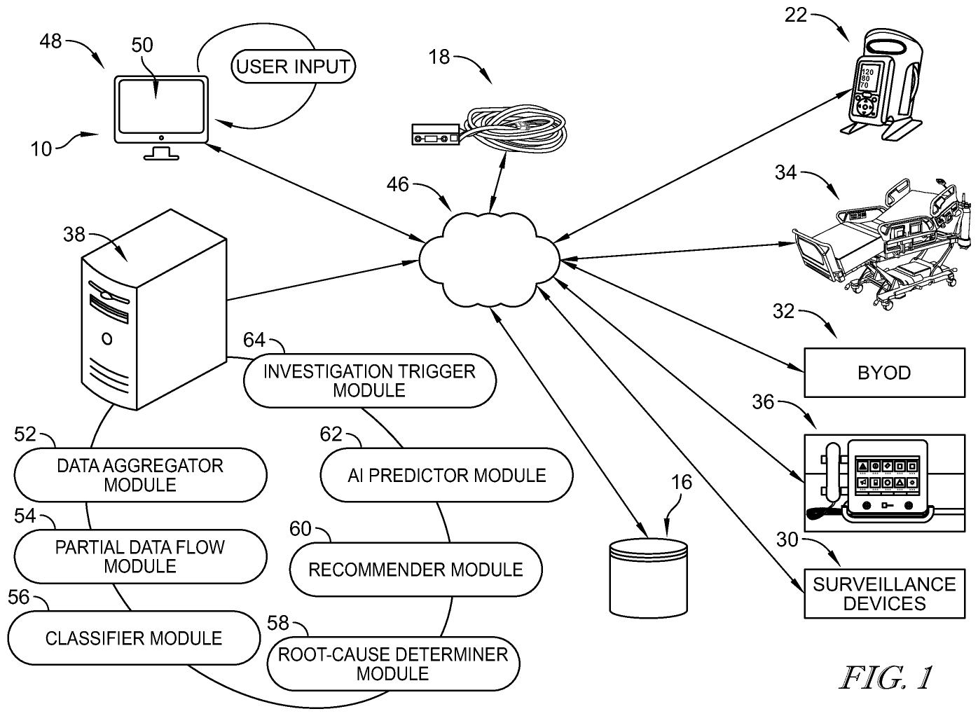

The risk evaluator 38 can includes a data aggregator module 52, a partial data flow module 54, a classifier module 56, a root-cause determiner module 58, a recommender module 60, an AI predictor module 62, and an investigation trigger module 64. As shown in FIG. 1, the risk evaluator 38 can analyze information from the one or more assessing devices 14 and executed one or more modules 52, 54, 56, 58, 60, 62, 64.

As shown in FIG. 1, the risk evaluator 38 can transmit reports, analysis, and/or other forms of output from the modules 52, 54, 56, 58, 60, 62, 64 to one or more user devices 48 in the form of alerts, charts, messages, and/or other audible and/or visual output. The user device 48 can include a display 50 and can illustrate risk associated with patient-care as evaluated by the control system 38.

The risk evaluation system 10 uses information received from various assessing devices 14 including but not limited to patient electronic medical record (EMR) 16, a sensor 18 (e.g., pressure sensors, temperature sensors, sound sensors, etc.), patient support devices 22 (e.g., a patient-controlled analgesia (PCA) pump 24, a respiratory equipment 26, a fluid management device 28), a surveillance device 30, a bring your own device (BYOD) 32, a patient support apparatus 34, and a real-time locating system (RTLS) 36 over a network 46. Additionally, one or more user devices 48 can be used to provide information to the risk evaluator 38 when evaluating the occurrence of a safety event. In some embodiments, user input and/or one or more sensors 18 can be used to detect a safety event.

In some embodiments, the risk evaluator 38 can include all modules 52, 54, 56, 58, 60, 62, 64. In other embodiments, the risk evaluator 38 can include one or more of the modules 52, 54, 56, 58, 60, 62, 64, but not all of the modules 52, 54, 56, 58, 60, 62, 64. In some embodiments, one or more modules 52, 54, 56, 58, 60, 62, 64 of the risk evaluator 38 can be operated or implemented automatically and/or in real-time. In some embodiments, one or more modules 52, 54, 56, 58, 60, 62, 64 of the risk evaluator 38 can be operated or implemented after user input or trigger.

The risk evaluator 38 can provide outputs (e.g., reports) at various stages of risk evaluation. For example, an incident investigation report can provide immediate access to a timeline of actions leading to and after the safety event. The incident investigation report can further include patient complaints, falls, violent events, patient deterioration, and other information. Similarly, a fall compliance report can be used to track bed settings for fall-risk patients, highlight bed settings that may put patients at risk, and strengthen fall prevention strategies by uncovering areas of improvement.

The risk evaluator 38 can provide both short-term and long-term reports. One or more of the short-term and long-term reports are transmitted to one or more user devices 48. For example, short-term reports can include a post-event report, an event investigation report, an event analysis and executive review, and/or an action plan. The post-event reporting outlines safety event response and an immediate mitigating strategy. The event investigation report can identify people involved, and provide an in-depth chart review and interviews of providers and caregivers. The event analysis and executive review can be directed to management and provide an analysis of root causes by assessing similar cases. The action plan can provide information about the next steps in risk mitigation. Long-term reports can include a mandated reporting list, an organization's safety metrics, a patient and staff satisfaction survey, and a report for tracking success. The mandated reporting list can include all state-level verification requirements and cross-checks.

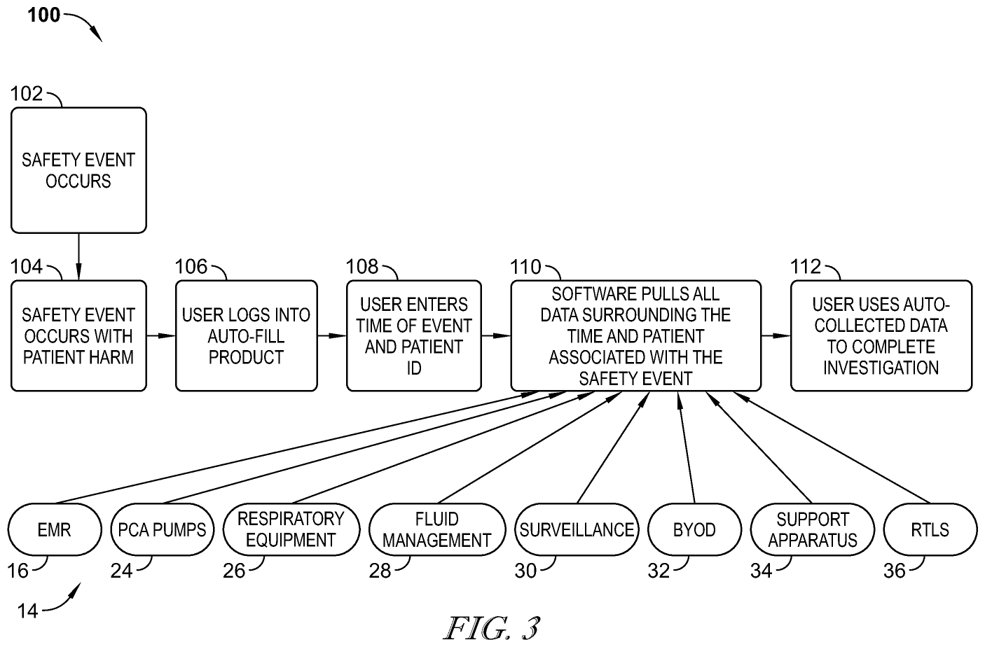

As shown in FIG. 3, the data aggregator module 52 can be executed after the occurrence of any safety event in step 102. The data aggregator module 52 can perform a set of operations 100 illustrated in FIG. 3. The data aggregator module 52 can access all relevant patient data from assessing devices 14 including but not limited to the patient electronic medical record (EMR) 16, the PCA pump 24, the respiratory equipment 26, the fluid management device 28, the surveillance device 30, the BYOD 32, the patient support apparatus 34, and the RTLS 36. If the safety event in step 102 is determined to cause patient harm in step 104, a user (e.g., caregiver) is prompted to log into an auto-fill product (e.g., a form, a table, etc.) in step 106 provided the user has the required permissions to access patient data. The user is prompted to enter a time of occurrence of the safety event and the patient identification number in step 108. The data aggregator module 52 can access all the collected data about that particular patient and identify specific event-related data during a given time-period surrounding the safety event in step 110. The user is prompted to access and utilize all the collected information from the data aggregator module 52 on one or more user devices 48 to complete an investigation into the safety event that occurred in step 112.

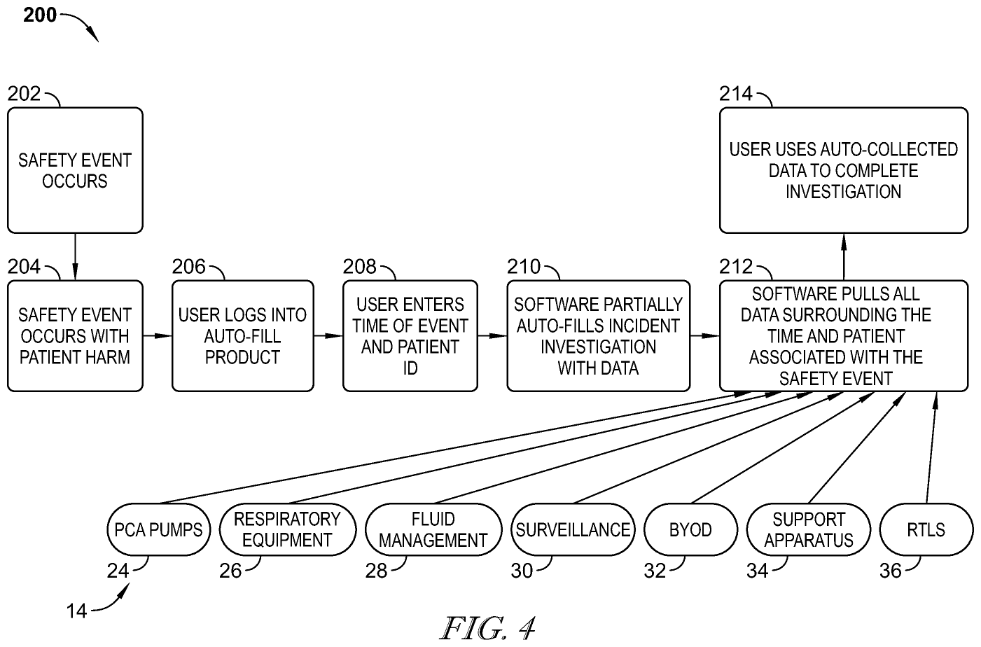

As shown in FIG. 4, the partial data flow module 54, can be executed after the occurrence of any safety event in step 202. The partial data flow module 54 can perform a set of operations 200 illustrated in FIG. 4. The partial data flow module 54 can access all relevant patient data from assessing devices 14 including but not limited to the PCA pump 18, the respiratory equipment 20, the surveillance devices 22, the fluid management devices 24, the BYOD 26, the patient support apparatus 28, and the RTLS 30. If the safety event in step 202 is determined to cause patient harm in step 204, a user is prompted to log into the auto-fill product in step 206 provided the user has the required permissions to access patient data. The user is prompted to enter the time of occurrence of the safety event and the patient identification number in step 208.

The partial data flow module 54 can partially autofill an incident investigation report with sample data points in step 210. The partial data flow module 54 can use information from the patient's electronic medical record (EMR) 16, patient support apparatus 34, and RTLS 36 to partially auto-fill the incident investigation report. The partial data flow 54 can access all the data collected by and/or utilized by the PCA pumps 24, respiratory equipment 26, surveillance devices 30, fluid management devices 28, BYOD 32, patient support apparatus 34, and RTLS 36 about that particular patient associated with the time of occurrence of the safety event in step 212. The user is prompted to access and utilize all the collected information from the partial data flow module 54 on one or more user devices 48 to complete an investigation into the safety event that occurred in step 214.

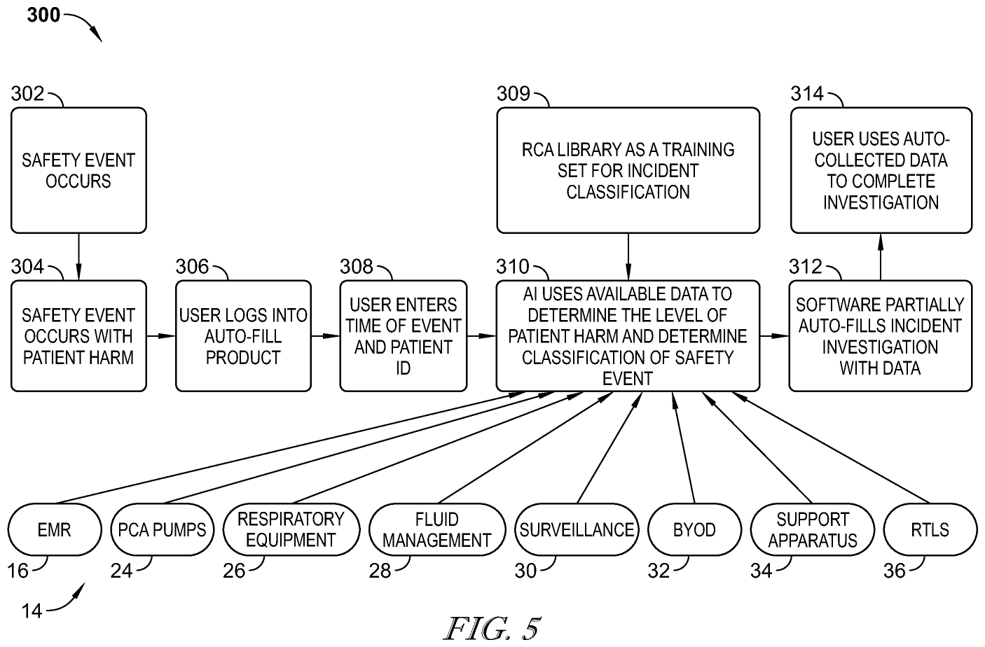

As shown in FIG. 5, the classifier module 56 can be executed after the occurrence of any safety event in step 302. The classifier module 56 can perform a set of operations 300 illustrated in FIG. 5. The classifier module 56 can access all relevant patient data from assessing devices 14 including but not limited to the patient electronic medical records 16, the PCA pump 24, the respiratory equipment 26, the surveillance device 30, the fluid management device 28, the BYOD 32, the patient support apparatus 34, and the RTLS 36. If the safety event in step 302 is determined to cause patient harm in step 304, a user is prompted to log into an auto-fill product in step 306 provided the user has the required permissions to access patient data. The user is prompted to enter the time of occurrence of the safety event and the patient identification number in step 308. The classifier module 56 can execute artificial intelligence (AI) based operations implemented by an AI system 42 to determine the level of patient harm and/or to determine a classification of the safety event in step 310. The AI-based operation can include machine learning techniques, neural networks, automation, and or other types of artificial intelligence. Prior to determining the level of patient harm and/or the classification of the safety event, the classifier module 56 utilizes a root cause analysis (RCA) library as a training set for incident classification in step 309.

The incident investigation can be auto-filled in all reports and/or forms in step 312 based on the analysis conducted in step 310. The user can access the collected data on one or more user devices 48. The user is prompted to use the collected data to complete the investigation in step 314.

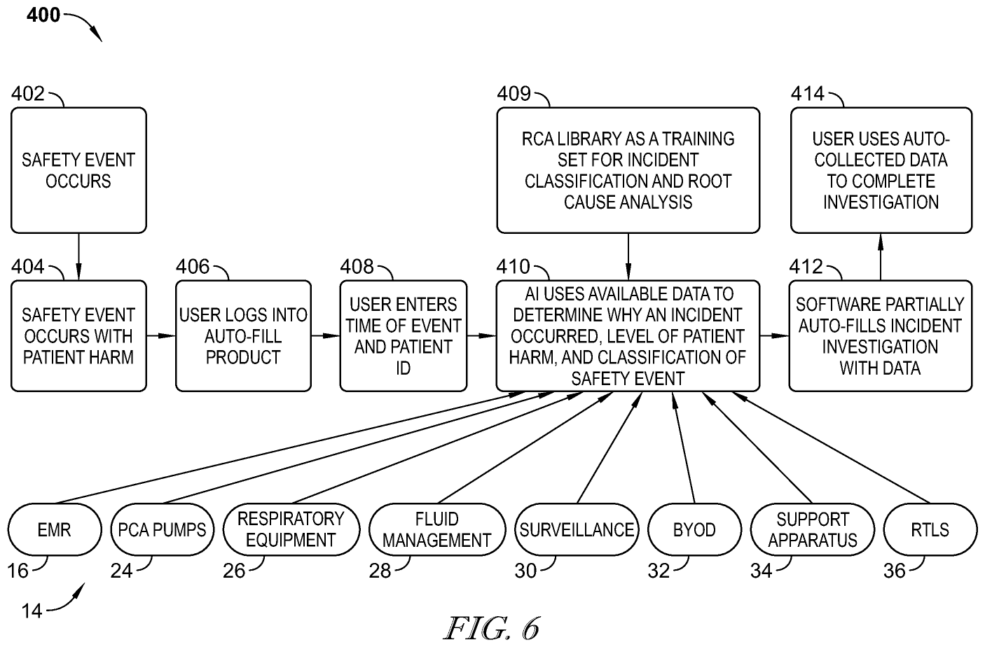

As shown in FIG. 6, the root-cause determiner module 58 can be executed after the occurrence of any safety event in step 402. The root-cause determiner module 58 can perform a set of operations 400 illustrated in FIG. 6. The root-cause determiner module 58 can access all relevant patient data from assessing devices 14 including but not limited to the patient electronic medical record (EMR) 16, the PCA pump 24, the respiratory equipment 26, the surveillance devices 30, the fluid management device 28, the BYOD 32, the patient support apparatus 34, and the RTLS 36. If the safety event in step 402 is determined to cause patient harm in step 404, a user can log into an auto-fill product in step 406 provided the user has the required permissions to access patient data. The user is prompted to enter the time of occurrence of the safety event and the patient identification number in step 408 on one or more user devices 48.

The root-cause determiner module 58 can execute AI-based operations implemented by the AI system 42 that access and utilize all data from assessing devices 14 to determine the cause of the safety event in step 410. Prior to determining the cause of the safety event in step 410, the root-cause determiner module 58 utilizes the RCA library as a training set for incident classification in step 409. The incident investigation can be auto-filled in all reports and/or forms in step 412 based on the analysis conducted in step 410. The user is prompted to access, confirm, and use the information provided to complete the investigation on one or more user devices 48 in step 414.

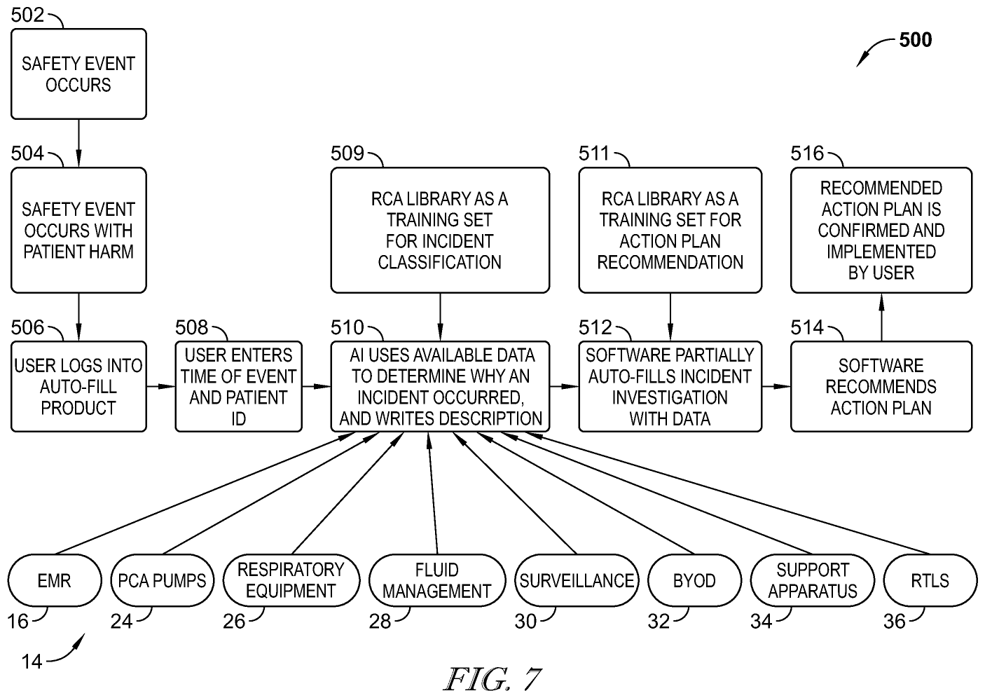

As shown in FIG. 7, the recommender module 60 can be executed after the occurrence of any safety event in step 502. The recommender module 60 can perform a set of operations 500 illustrated in FIG. 7. The recommender module 60 can access all relevant patient data from assessing devices 14 including but not limited to the patient electronic medical record (EMR) 16, the PCA pump 24, the respiratory equipment 26, the surveillance device 30, the fluid management device 28, the BYOD 32, the patient support apparatus 34, and the RTLS 36. If the safety event in step 502 is determined to cause patient harm in step 504, a user can log into an auto-fill product in step 506 provided the user has the required permissions to access patient data. The user is prompted to enter the time of occurrence of the safety event and the patient identification number in step 508. The recommender module 60 can execute AI-based operations implemented by the AI system 42. The AI-based operations can access and utilize all data from assessing devices 14 to determine the cause of the safety event and to provide a written description in step 510. Prior to determining the cause of the safety event and providing a written description in step 510, the recommender module 60 utilizes the RCA library as a training set for incident classification in step 509.

The incident investigation can be auto-filled in all reports and/or forms in step 512 based on the analysis conducted in step 510. The recommender module 60 can recommend an action plan to the user in step 514. Prior to recommending an action plan in step 514, the recommender module 60 utilizes the RCA library as a training set for action plan recommendation in step 511. After a recommendation is provided, the user can access and confirm the recommended action plan on one or more user devices 48. The user can implement the recommended action plan in step 516

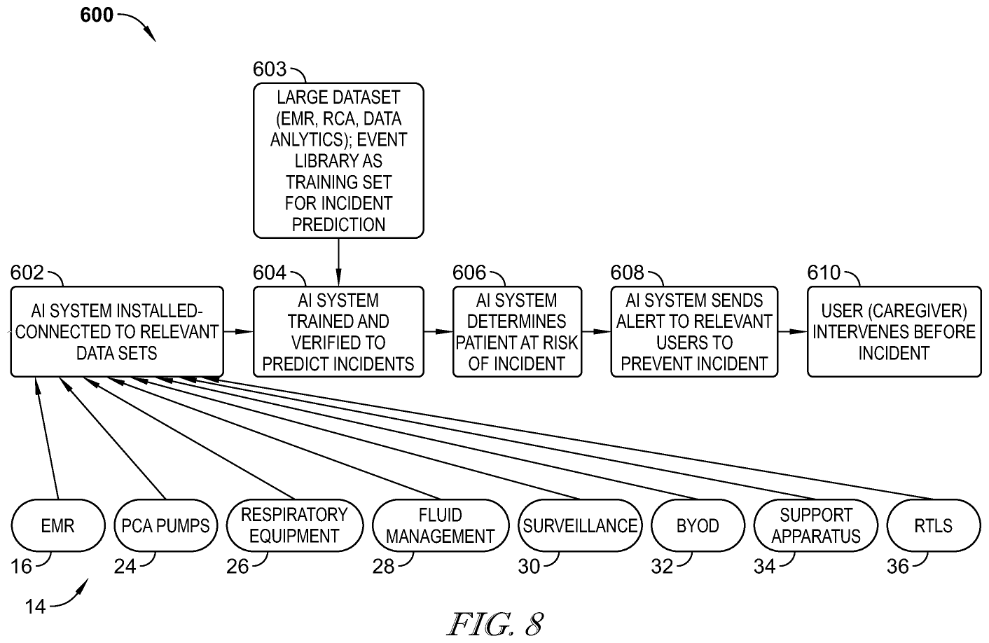

The AI predictor module 62 can be executed individually or in conjunction with any other module in the risk evaluation system 10. The AI predictor module 62 can perform a set of operations 600 illustrated in FIG. 8. As shown in FIG. 8, after the AI system 42 is installed and connected to the relevant data sets in step 602, the predictor module 62 can access all relevant patient data from assessing devices 14 including but not limited to the patient electronic medical record (EMR) 16, the PCA pump 24, the respiratory equipment 26, the surveillance device 30, the fluid management device 28, the BYOD 32, the patient support apparatus 34, the RTLS 36.

The AI system 42 is trained and verified to be able to predict incidents (e.g., safety events) in step 603. The AI system 42 utilizes large data sets including data from the EMR 16, recommended action plans, and other data analytics for training in incident prediction. After being trained, the AI system 42 is operated or implemented to determine if any patient is at risk of any safety-related incident in step 606. If the AI system 42 ascertains that a particular patient is at risk, the AI system 42 transmits an alert to any relevant user to prevent the occurrence of the safety-related incident in step 608. The alert can be a visual and/or an audible alert. The alert can be sent to an individual user (e.g., caregiver associated with the patient), or to multiple users (e.g., caregivers in the vicinity of the patient). The alert can be sent to one or more personal computational devices of one or more users. Once the alert is intercepted, the user can intervene before the safety-related incident occurs in step 610.

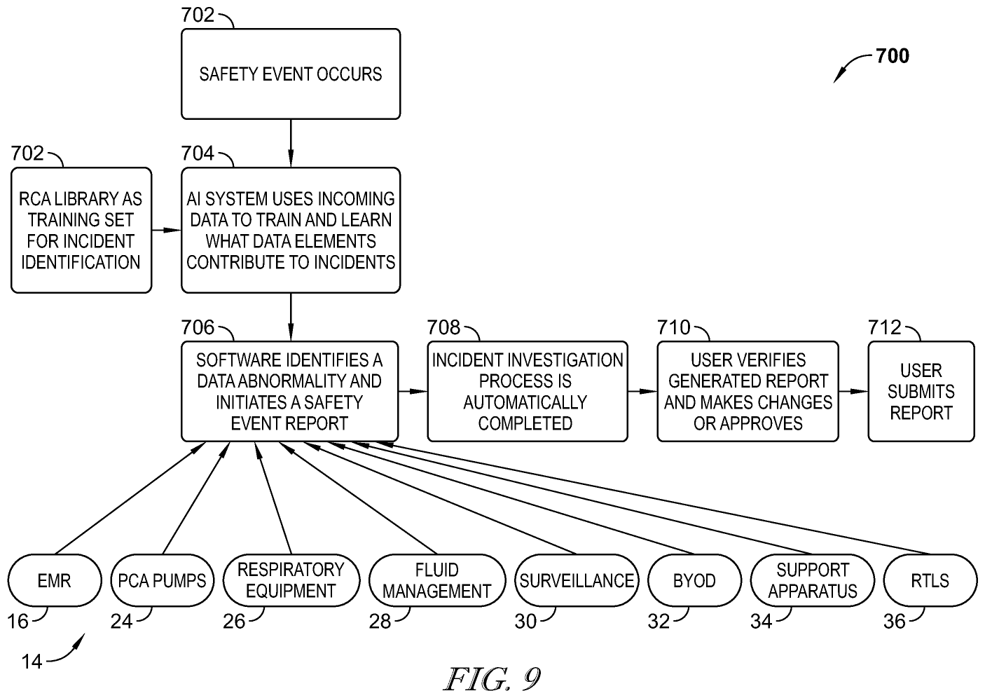

As shown in FIG. 9, the investigation trigger module 64 can be executed after the occurrence of any safety event in step 702. The investigation trigger module 64 can perform a set of operations 700 illustrated in FIG. 9. The AI system 42 uses incoming data to train its incident predictive capability in step 704. The AI system 42 learns how data elements can contribute to safety-related incidents. The AI system 42 uses the RCA library as a training set for incident identification in step 702. The investigation trigger module 64 can access all relevant patient data from assessing devices 14 including but not limited to the patient electronic medical record (EMR) 16, the PCA pump 24, the respiratory equipment 26, the surveillance device 30, the fluid management device 28, the BYOD 32, the patient support apparatus 34, and the RTLS 36. Software in the investigation trigger module 64 identifies data abnormality and initiates a safety event report in step 706.

An incident investigation process is automatically completed by the investigation trigger module 64 in step 708. The user is prompted to verify the generated safety event report and make changes to the report in step 710. The user can approve the report without making any changes in step 710. After approval, the user is prompted to submit the generated report in step 712.

Information obtained from the risk evaluation system 10 can be leveraged in different reports including but not limited to reports about bed status, event chronology, staff activity, and nurse call details to comprehend occurrences leading up to the safety even. For example, reports about staff activity can clarify which caregiver responded to a fall, when a badged caregiver entered the room, or what rounds were completed at the time of the safety event. Bed setting of a patient can be determined based on the bed setting history, evaluating a falls risk scorecard that associates the risk of falling with different variables, and a bed status report. Caregiver calls can be monitored by tracking response time to reduce patient wait times and ensure that the caregiver reacts appropriately to patient needs.

Although this disclosure refers to specific embodiments, it will be understood by those skilled in the art that various changes in form and detail may be made without departing from the subject matter set forth in the accompanying claims.

1. A method for evaluating risk associated with a patient comprising the steps of:
detecting, using a sensor or an assessing device, a safety event involving the patient,
acquiring, using the sensor or the assessing device, data for identifying and investigating the safety event,
receiving and processing the data to evaluate the risk associated with patient care,
operating a module of a risk evaluator to use the data acquired to determine an output of the risk associated with the patient, and
transmitting the output to a user device.

2. The method of claim 1, wherein operating the module of the risk evaluator comprises:
operating a data aggregator module comprising the steps of
determining if the safety event caused harm to the patient,
prompting a user to log a time of occurrence of the safety event,
identifying event-related data acquired from the sensor or the assessing device about the patient during a given time period based on the time of occurrence of the safety event, and
prompting the user to complete an investigation into the safety event.

3. The method of claim 1, wherein operating the module of the risk evaluator comprises:
operating a partial data flow comprising the steps of
determining if the safety event caused harm to the patient,
prompting a user to log a time of occurrence of the safety event,
partially auto-filling an incident investigation report based on the data received from the sensor and the assessing device,
identifying event-related data about the patient during a given time period based on the time of occurrence of the safety event, and
prompting the user to complete an investigation into the safety event by providing the partially auto-filled incident investigation report.

4. The method of claim 1, wherein operating the module of the risk evaluator comprises:
operating a classifier comprising the steps of
determining if the safety event caused harm to the patient,
prompting a user to log a time of occurrence of the safety event,
utilizing an AI-based operation to determine a classification of the safety event,
partially auto-filling an incident investigation report with the data received from the sensor and the assessing device, and
prompting the user to complete an investigation into the safety event by providing the partially-filled incident investigation report.

5. The method of claim 1, wherein operating the module of the risk evaluator comprises:
operating a root-cause determiner module comprising the steps of
determining if the safety event caused harm to the patient,
prompting a user to log a time of occurrence of the safety event,
utilizing an AI-based operation to determine a root-cause of the safety event,
partially auto-filling an incident investigation report based on the data received from the sensor and the assessing device, and
prompting the user to complete an investigation into the safety event by providing the partially auto-filled incident investigation report.

6. The method of claim 1, wherein operating the module of the risk evaluator comprises:
operating a recommender module comprising the steps of
determining if the safety event caused harm to the patient,
prompting a user to log a time of occurrence of the safety event,
utilizing an AI-based operation to determine a root-cause of the safety event,
utilizing the AI-based operation to write a description about the root- cause of the safety event,
partially auto-filling an incident investigation report with the data received from the sensor and the assessing device, and
recommending an action plan to the user based on the root-cause determination made by the AI-based operation.

7. The method of claim 6, wherein the AI-based operation is configured to access data from a patient-controlled analgesia (PCA) pump, a respiratory equipment, a surveillance device, a fluid management device, a bring your own device (BYOD), a patient support apparatus, or a real-time locating system (RTLS).

8. The method of claim 1, wherein operating the module of the risk evaluator comprises:
operating an AI predictor module comprising the steps of
installing and connecting an AI system to the accessing device,
training and verifying the AI system's ability to predict a second safety event,
utilizing the AI system to predict the second safety event, and
transmitting an alert to the user to prevent an occurrence of the second safety event.

9. The method of claim 1, wherein operating the module of the risk evaluator comprises:
operating an investigation trigger module comprising the steps of
training an AI system to learn about data elements that contribute to the safety event,
identifying a data abnormality from the data received from the sensor and assessing device,
completing an incident investigation process and generating a safety event report, and
transmitting the safety event report to the user device.

10. A method for evaluating risk associated with a patient comprising the steps of:
receiving and processing data to evaluate the risk associated with patient care,
operating a module of a risk evaluator to use the data acquired to determine an output of the risk associated with the patient, and
transmitting the output to a user device.

11. The method of claim 10, wherein operating the module of the risk evaluator comprises utilizing AI-based operation to determine a level of patient harm.

12. The method of claim 6, wherein the AI-based operation is configured to access data from a patient-controlled analgesia (PCA) pump, a respiratory equipment, a surveillance device, a fluid management device, a bring your own device (BYOD), a patient support apparatus, or a real-time locating system (RTLS).

13. A system for evaluating risk associated with a patient comprising:
an assessing device to capture data for identifying and investigating a safety event,
a risk evaluator including a controller and a memory device, the controller operable to receive and process data from the assessing device, the controller further operable to evaluate the risk associated with patient care by operating a module based on the data and a training dataset in the memory device, and
a user device including a display, the display illustrating risk associated with patient-care.

14. The system of claim 13, wherein the accessing device comprises a patient-controlled analgesia (PCA) pump, a respiratory equipment, a surveillance device, a fluid management device, a bring your own device (BYOD), a patient support apparatus, or a real-time locating system (RTLS).

15. The system of claim 13, wherein the risk evaluator is operable to implement a data aggregator module, a partial data flow module, a classifier module, a root-cause determiner module, a recommender module, an AI predictor module, or an investigation trigger module.

16. The system of claim 13, wherein the risk evaluator further comprises a AI system, and wherein the risk evaluator is operable to implement a classifier module configured to access data about the patient from the assessing device during a given time period based on a time of occurrence of the safety event, utilize an AI-based operation to determine a classification of the safety event, and partially auto-fill an incident investigation report with the data.

17. The system of claim 13, wherein the risk evaluator further comprises a AI system, and wherein the risk evaluator is operable to implement a root-cause determiner module configured to access data about the patient from the assessing device during a given time period based on a time of occurrence of the safety event, utilize an AI-based operation to determine a root-cause of the safety event, and partially auto-fill an incident investigation report with the data.

18. The system of claim 13, wherein the risk evaluator further comprises an AI system, and wherein the risk evaluator is operable to implement a recommender module configured to access data about the patient from the assessing device during a given time period based on a time of occurrence of the safety event, utilize an AI-based operation to determine a root-cause of the safety event, partially auto-fill an incident investigation report with the data, and transmit a recommended action plan to the user device.

19. The system of claim 13, wherein the risk evaluator further comprises an AI system connected to the assessing device, and wherein the risk evaluator is operable to implement an AI predictor module configured to predict a second safety event and transmit the prediction to the user device.

20. The system of claim 13, wherein the risk evaluator further comprises an AI system, and wherein the risk evaluator is operable to implement an investigation trigger module configured to identify a data abnormality from the data received from the assessing device, complete an incident investigation process, generate a safety event report, and transmit a the safety report to the user device.
